(19) United States
(12) Patent Application Publication
(10) Pub. No.: US 2009/0030378 A1
(43) Pub. Date: Jan. 29, 2009

(54) Medical in-line flow control clamp device

(76) Inventor: Raul Garcia, JR., El Paso, TX (US)

Correspondence Address: Faier & Faier P.C., 566 W. Adams St. #600, Chicago, IL, 60661, US

(21) Appl. No.: 12/070,490

(22) Filed: Feb. 20, 2008

Related U.S. Application Data
(60) Provisional application No. 60/962,252, filed on Jul. 25, 2007.

(51) Int. Cl. A61M 5/00

(52) U.S. Cl. 604/249; 604/250

(57) ABSTRACT

A medical in-line flow control clamp device which is arranged coaxially along the longitudinal axis of an infusion tube or the like, and which has a sleeve connected to the tube and a slide having an arm movably mounted on and having a part biased toward the sleeve in a manner where the arm bears against and pinches the tube when the slid is moved relative to the sleeve. The sleeve may have a ramp and support directed toward the tube which causes the arm to move against the tube. The slide may be fabricated from a plastic material, such as acrylic, which causes the arm to bind against the tube. The sleeve may have one or more stops and latch means to receive and secure the arm in tube pinching relation. The sleeve and slide may have a track and projection parts which train the slide in a predetermined direction toward the tube as the slide is moved to pinching position on the sleeve. Bars may be arranged on the slide transverse to the sleeve and tube for manipulating the slide on the sleeve by finger pressure. The sleeve may be fabricated in portions connected by a web so they may be folded and held around the tube in a selected position.

### PRIORITY CLAIM

Priority is claimed based upon U.S. Provisional Patent Application Ser. No. 60/962,252, MEDICAL IN-LINE FLOW CONTROL CLAMP DEVICE, filed Jul. 5, 2007.

### MEDICAL IN-LINE FLOW CONTROL CLAMP DEVICE

This invention relates to medical in-line flow control devices, and is more particularly concerned with such devices having the capability to regulate flow of fluid in a tube used for medical purposes, and a device which is not substantially enlarged as to encumber the appearance or mobility of the persons requiring delivery of fluid through or from the tube.

### BACKGROUND AND SUMMARY OF THE INVENTION

Most conventional flow control devices are of one of several types:

1. Large one piece clips or clamps, exemplified by those shown in Flynn U.S. Pat. Nos. 4,673,161 or 4,588,160, which have been refined over the years with various locking arrangements, as in Guala et al U.S. Pat. No. 6,161,812 and US Patent Application 2006/0079849

2. Devices which clamp a tube laterally to its longitudinal direction, exemplified by those shown in Jacobson U.S. Pat. No. D283,918 or Miller Jr. U.S. Pat. No. 3,461,876, which have been refined by locking and securing members, as in Porat U.S. Pat. No. 6,234,448,

3 Roller clamps which bear against the tube as in D'Alessio et al U.S. Pat. No. 5,259,587 or Konzak U.S. Pat. No. 3,289,999, which have been improved with such structure as non-linear roller clamp bodies, such as those shown in Height et al U.S. Pat. No. 6,929,236.

While these and other variations of these flow control devices for flexible plastic tubes used for delivering fluid to or removing fluid from a patient, all these known or conventional devices have a mass substantially larger than the fluid tube, and where the patent is ambulatory or dressed, the clamp shows through the patient's garment and its use is inconvenient or embarrassing for the patient or relatively easy to dislodge.

A device embodying the present invention is not only efficient for controlling flow of liquids in the tube to which it is attached, but it is structured in line with the longitudinal axis of the tube and is substantially no larger or substantially more offset from the wearer than the tube, so it does not show through garments and it does not present an enlarged cross section which can be inconvenient or embarrassing for the patent or becomes insecure. Further, where the patient may lay upon the device, the presence of the clamp is no more painful, inconvenient or stressful than the tube itself as it will lie against the patient's body in line with the tube.

Additionally, the locking device of the preferred embodiment of the invention presents a positive closure, which requires action for opening the tube, and is not easily dislodged as is often the case with conventional tube clamps, as evidence by the presence in the art of so many locking and securing structures.

The device embodying the present invention also can be manipulated with one hand by finger pressure, so that a person using the device can easily open or close the device with mere finger movement, while some clamps require two hands to manipulate.

### OBJECTS AND ADVANTAGES OF THE INVENTION

It is the object of this invention to provide a novel in-line flow control device for medical use of the character described.

Another object is to provide a medical in-line flow control device which will lay flat against an infusion tube to which it is attached for use in opening or closing an infusion tube.

Another object is to provide a medical flow control device for opening and closing an infusion tube which may be arranged along the longitudinal axis of the tube.

Another object is to provide a medical in-line flow control device which permits positive opening and closing of an infusion tube to which it may be attached.

Another object is to provide a medical in-line flow control device which inhibits unintentional opening or closing.

Another object is to provide a locking and unlocking lug for an in-line medical flow control device.

Another object is to provide a track for guiding an in-line medical flow control device into open or closing position.

Another object is to provide a flow control device which can be installed on an infusion tube intermediate its length.

Another object is to provide an on-line flow control device which can be positively manipulated to control the volume or speed of inflow of fluid into or from the device.

Another object is to provide an in-line flow control device for a medical infusion tube which can be molded from plastic, and which is easy and expedient to use and efficient to manufacture.

These and other objects and advantages of the invention will become more apparent as this description proceeds, taken in conjunction with the accompanying drawings.

### BRIEF DESCRIPTION OF THE DRAWINGS

In the drawings:

FIG. 1 is a schematic view showing a typical use of an in-line medical flow control device embodying the present invention.

FIG. 2 is a perspective view of the device embodying the present invention secured on a tube in open condition to permit a flow of fluid through the tube.

FIG. 3 is a perspective view of the device embodying the present invention secured on a tube similar to FIG. 2, except in closed condition to inhibit a flow of fluid through the tube.

FIG. 4 is sectional view of clamping device shown in FIG. 2, in open condition.

FIG. 5 is sectional view of clamping device shown in FIG. 3, in closed condition.

FIG. 6 is an elevational view of a sleeve which may be fabricated in portions and folded together around a tube.

FIG. 7 is an elevational view of part of the slide.

FIG. 8 is an elevational view of the sleeve shown in FIG. 6 taken at a right angle thereto.

FIG. 9 is a sectional view of the sleeve takedm on line 9-9m of FIG. 7.

### DESCRIPTION OF PREFERRED EMBODIMENTS

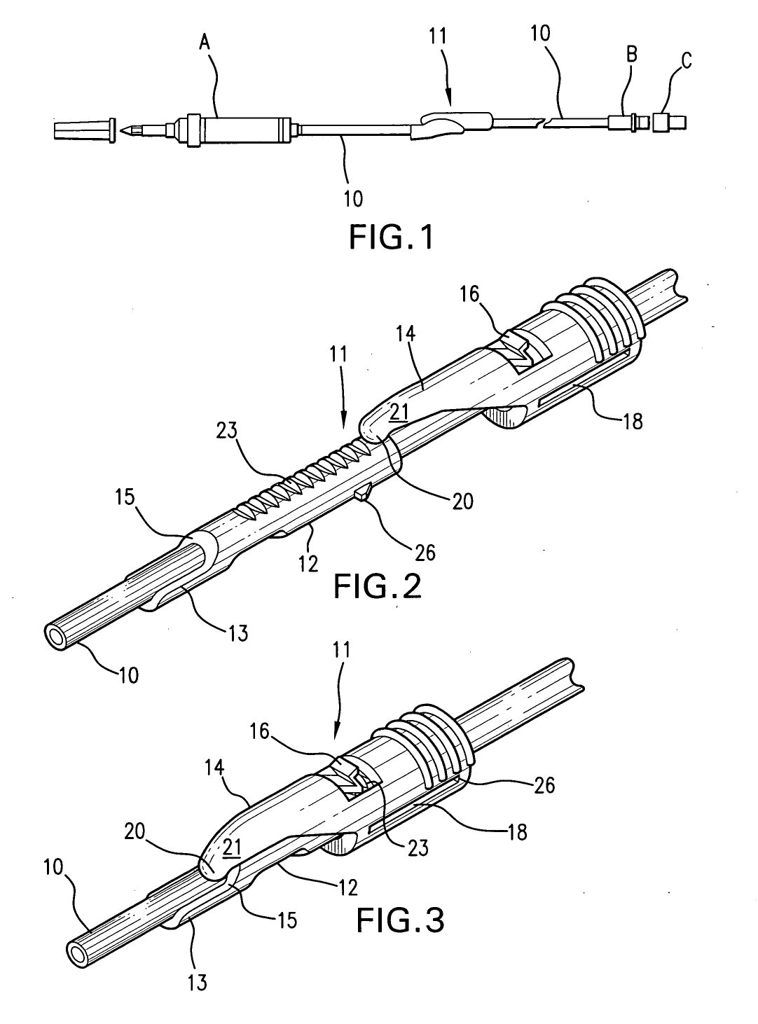

With reference to FIG. 1, a typical use of a flow control device embodying the present invention is in an intravenous administration set which may include a IV chamber A, an infusion tube 10, a clamp 11, a slip B and a cap C. Similar arrangements of a tube 10 and clamp 11 may be used in transfusion sets or in other programs for delivering medicament to or withdrawing fluid from a patient. The clamp 11 may be opened to permit flow of medicament or fluid through the tube 10 or closed to prevent flow. Similar arrangements are used in withdrawing fluid from the body of the patient, in which case the tube 10 is opened to permit flow and closed when a tube must be changed or other action is required. A Clamp 11 embodying the present invention may be utilized on tubes in all environments which presently use conventional roller clamps or foldable or static clips or clamps.

Preferably, flow control devices embodying the present invention are constructed from plastic material, such a polypropylene, nylon or polyethylene, but they may also be wholly or in part fabricated from acrylic materials; and they should be relatively flexible. Such devices are preferably molded. The clamp 11 which embodies the present invention comprises a first component which consists of a sleeve 12 adapted for arrangement longitudinally, coaxially on an infusion tube 10 and a second component which consists of a slide 14 arranged longitudinally, coaxially on the sleeve 12. The sleeve 12 has a ramp 15 near its forward edge. The slide 14 is slidably engaged over the outside circumference of the sleeve 12, and has an arm 21 arranged extending from the slide 14 in line with an anvil or support 13 extending from the sleeve 12.

The arm 21, which is normally biased toward the support 13, has on its free end forward edge an enlarged detent 20 adapted to slide down the ramp 15 as the slide 14 is moved forward relative to the sleeve 12. The slide 12 may be molded from acrylic plastic and the sleeve 14 may be molded from poly vinyl chloride (pvc) material, in which case the slide arm 21 and detent 20 tends to bind and remain positioned where placed on the sleeve until moved, so the user can control the pinching action of the slide on the sleeve 12 to adjust the flow of fluid through the tube 10.

Tto encourage smooth action of the slide 14 on the sleeve 12 projections 26 may be interfitted with tracks 18 on the mating surfaces of the slide 14 and sleeve 12 to direct and control movement of the slide on the sleeve for properly placing the detent 20 of the arm 21 on the tube 10. Also, for positively modulating flow through the tube 10, one or more stops 23 which may be engaged by a locking lever 16 on the slide 14 to engage the sleeve 12 in a predetermined position to partially or fully pinch the tube 10. Easy gripping of the slide 14 by the fingers or thumb of one hand by mere finger pressure is enhanced by manipulating the bars 19 spaced along the periphery of the slide.

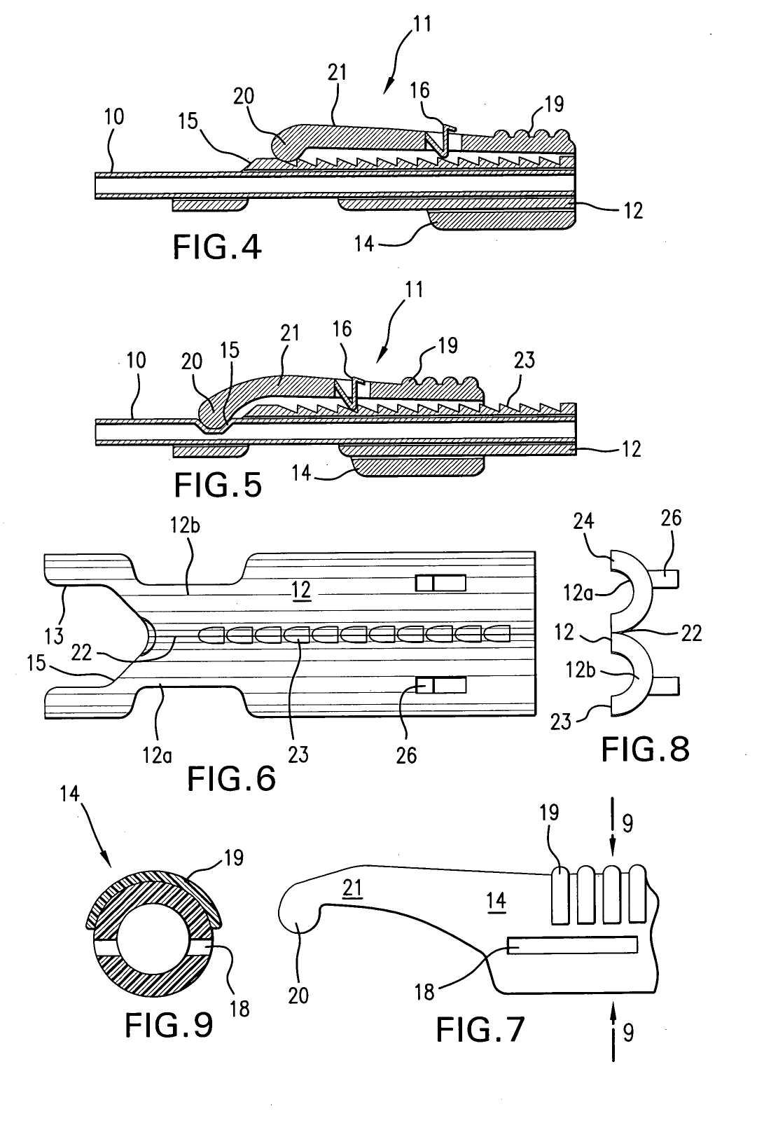

In the embodiment disclosed in FIGS. 2-5, the sleeve 12 is arranged over the tube 10, preferably near its end adjacent connection to the patient. To open or close the tube 10, the slide 14 is slid forward relative to the sleeve 12 until the arm 21 and its enlarged detent 20 moves down the ramp 15 to pinch-engage the tube 10 over the support 13. Flow may also be controlled or modulated by fabricating the sleeve from plastic such as nylon and the slide from acrylic material, which permits the slide 14 to be positioned and held at any point along the ramp 15 to modulate the flow of fluid through the tube 10.

The flow control clamp 11 may be attached at any point along the tube 10. The sleeve 12 may be fabricated in parts which are foldable toward one another along a connecting web 22 and turned together to embrace the tube 10. As shown in FIGS. 6 and 8, the sleeve portions 12a and 12b may snap engaged and secured together by nibs 23 on one portion 12b and nib receivers 24 (shown in fantom) on the other portion 12a.

While preferred embodiments of the invention have been shown in considerable detail, many changes and modifications may b made without departing from the spirt or scope of the invention. Accordingly, it is not desired that the invention should be limited to the exact construction shown and described.

1. A medical flow control device for delivering fluid to or from a patient through a tube, said device comprising a sleeve adapted to be axially aligned with and lay over said tube, said sleeve having a cut away portion, and a slide axially aligned with and arranged over said sleeve and said tube in line with said sleeve and said tube, said sleeve having an arm adapted to engage said cut away portion of said sleeve and pinch said tube when arranged over said cut away portion and permit said tube to remain open when said arm is not arranged over said cut away portion.

2. The medical flow control device recited in claim 1, wherein said sleeve cut away portion is inclined toward said tube and defines a ramp for movement of said arm relative to said tube.

3. The medical flow control device recited in claim 2, wherein said arm has an enlarged detent on a free end adapted to engage said tube over said cut away portion.

4. The medical flow control device recited in claim 2, wherein said arm is biased toward said cut away portion.

5. The medical flow control device recited in claim 1, wherein said sleeve has stop means for locating said arm at selected position relative to said cut away portion.

6. The medical flow control device recited in claim 5, wherein said stop means comprises multiple spaced apart stops on it periphery remote from said tube and angularly arranged relative to the longitudinal axis of said tube.

7. The medical flow control device recited in claim 5, wherein said stop means has latch means for engaged said stop means to secure said stop means in a selected position.

8. The medical flow control device recited in claim 1, wherein said device is fabricated in one piece from plastic material.

9. The medical flow control device recited in claim 8, wherein said sleeve is fabricated from a different plastic than said slide, and said slide binds upon said sleeve until manipulated by hand pressure toward said cut away portion.

10. The medical flow control device recited in claim 1, wherein in said sleeve is fabricated in multiple parts connected by a web.

11. The medical flow control device recited in claim 10, wherein said parts are secured together around said tube to form said slide.

12. The medical flow control device recited in claim 1, wherein said device is not as thick as it is long.

13. The medical flow control device recited in claim 1, wherein the periphery of said slide remote from said sleeve has a bar arranged transverse to the longitudinal axis of said tube which may be manipulated to move said slide relative to said sleeve.

14. A process for clamping or unclamping an infusion tube to respectively open or close said tube, said process comprising the steps of
(1) attaching a sleeve having pinch receiving means in line with the longitudinal axis of said tube;
(2) connecting a slide having tube pinching means over and in line with said sleeve pinch receiving means along the longitudinal axis of said tube and said sleeve; and
(3) moving said slide relative to said sleeve along the longitudinal axis of said tube and said sleeve so that said slide pinching means pinches said sleeve pinch receiving means to close said tube or, alternatively, to withdraw said slide pinching means from said sleeve pinch receiving means to open said tube.

15. The process for clamping or unclamping an infusion tube to respectively open or close
said tube as recited in claim 14, with the additional step of guiding said slide along said tube in a predetermined path to open or close said tube.

16. The process for clamping or unclamping an infusion tube to respectively open or close said tube as recited in claim 14, with the additional step of
manipulating said slide to open or close by means of finger pressure on bars arranged on the periphery of said slide.

17. The process for clamping or unclamping an infusion tube to respectively open or close said tube as recited in claim 14, with the additional step of providing latch means on said slide and said sleeve to modulate the opening or closing of said tube in a predetermined manner.

18. The process for clamping or unclamping an infusion tube to respectively open or close said tube as recited in claim 17, with the additional step of latching or unlatching said sleeve and slide to open or close said tube.
